(19) United States
(12) Patent Application Publication
(10) Pub. No.: US 2010/0145255 A1
(43) Pub. Date: Jun. 10, 2010

(54) System And Method For Reducing The Appearance Of Fine Lines And Wrinkles And Improving the Skin Tone

(76) Inventors: Lavinia C. Popescu, Jackson Heights, NY (US); Geoffrey Hawkins, Yardley, PA (US); Jack Lombardi, Massapequa, NY (US); Barbara Anne Wolf, Scarsdale, NY (US)

Correspondence Address: THE ESTEE LAUDER COS, INC, 155 PINELAWN ROAD, STE 345 S, MELVILLE, NY, 11747, US

(21) Appl. No.: 12/620,647

(22) Filed: Nov. 18, 2009

Related U.S. Application Data
(60) Provisional application No. 61/120,499, filed on Dec. 8, 2008.

(51) Int. Cl. A61M 35/00; A61K 31/197; A61K 31/7076; A61K 38/08; A61K 31/22; A61P 17/00

(52) U.S. Cl. 604/20; 514/565; 514/47; 514/17; 514/547

(57) ABSTRACT

The present invention provides a system for rejuvenating the skin, reducing the appearance of fine lines and wrinkles, and improving the skin tone, which includes both a topical composition and a phototherapy device. Specifically, the topical composition contains one or more skin rejuvenating agents selected from the group consisting of creatine, adenosine phosphate, acetyl carnitine, acetyl hexapeptide, and combinations thereof in a pharmaceutically or cosmetically acceptable carrier. The phototherapy device contains one or more light emitting diodes (LEDs) for emitting electromagnetic radiation effective for energizing the skin cells and enhancing skin absorption of the skin rejuvenating agents. More particularly, the topical composition is capable of transmitting at least 80% of the electromagnetic radiation emitted by the phototherapy device to the skin.

This application claims benefit of U.S. provisional application 61/120,499, filed Dec. 8, 2009.

### FIELD OF THE INVENTION

The present invention relates in general to system and method for rejuvenating the skin. More specifically, the present invention relates to system and method for reducing the appearance of fine lines and wrinkles and enhancing evenness of the skin tone.

### SUMMARY OF THE INVENTION

In one aspect, the present invention relates to a system for rejuvenating the skin, reducing the appearance of fine lines and wrinkles and improving the skin tone, which comprises:
(a) a topical composition comprising one or more skin rejuvenating agents selected from the group consisting of creatine, adenosine phosphate, acetyl carnitine, acetyl hexapeptide, and combinations thereof in a pharmaceutically or cosmetically acceptable carrier; and
(b) a phototherapy device comprising one or more light emitting diodes (LEDs) for emitting electromagnetic radiation effective for energizing the skin cells and enhancing skin absorption of the skin rejuvenating agents,
   wherein when applied to the skin, the topical composition is capable of transmitting at least 80% of the electromagnetic radiation emitted by the phototherapy device to the skin.

In another aspect, the present invention relates to a unit package that comprises:
(a) a topical composition comprising one or more skin rejuvenating agents selected from the group consisting of creatine, adenosine phosphate, acetyl carnitine, acetyl hexapeptide, and combinations thereof in a pharmaceutically or cosmetically acceptable carrier; and
(b) a phototherapy device comprising one or more light emitting diodes (LEDs) for emitting electromagnetic radiation effective for energizing the skin cells and enhancing skin absorption of the skin rejuvenating agents,
   wherein when applied to the skin, the topical composition is capable of transmitting at least 80% of the electromagnetic radiation emitted by the phototherapy device to the skin.

In yet another aspect, the present invention relates to a method for rejuvenating the skin, reducing the appearance of fine lines and wrinkles and improving the skin tone, which comprises:
(a) applying to the skin a topical composition comprising one or more skin rejuvenating agents selected from the group consisting of creatine, adenosine phosphate, acetyl carnitine, acetyl hexapeptide, and combinations thereof in a pharmaceutically or cosmetically acceptable carrier; and
(b) radiating the skin with electromagnetic radiation emitted from one or more light emitting diodes (LEDs), wherein said electromagnetic radiation is effective for energizing the skin cells and enhancing skin absorption of the skin rejuvenating agents,
   wherein when applied to the skin, the topical composition is capable of transmitting at least 80% of the electromagnetic radiation emitted by the LEDs to the skin.

Other aspects and objectives of the present invention will become more apparent from the ensuing description, examples, and claims.

### DETAILED DESCRIPTION OF THE INVENTION, AND PREFERRED EMBODIMENTS THEREOF

The present invention uses light emitting diodes (LEDs) in combination with a topical composition containing skin care actives to achieve improved results in skin rejuvenation. Specifically, the topical composition contains skin a specific group of skin rejuvenating agents that are known to be effective in energizing the skin cells, stimulating collagen synthesis in the skin cells, boosting the self-repair of the skin cells, and ultimately reducing the appearance of fine lines and wrinkles and improving the skin tone. It is believed that the electromagnetic radiation (hereinafter “EM,” which includes, but is not limited to, visible light) emitted by the LEDs functions to energize skin cells and stimulate skin tissue in the deeper dermis layer, thereby enhancing absorption of the skin rejuvenating agents by the deep layer skin tissue. In order to fully effectuate the synergistic effect between the skin rejuvenating agents and the EM emitted by LED, the topical composition of the present application is specifically designed with a high optical transparency, so as to allow maximum amount of EM emitted by LED to be transmitted therethrough onto the skin surface. As used herein, the term “transmit” or “transmission” refers to passive and/or active conveyance of the EM through the topical composition onto the skin surface.

The LEDs as used in the present invention may emit EM in any wavelength. Preferably, the LEDs emit EM with a peak wavelength ranging from about 570 nm to about 850 nm. More preferably, the LEDs emit EM with a peak wavelength selected from the group consisting of about 580 nm, 630 nm, 633 nm, 660 nm and 830 nm and a bandwidth of no greater than +/−40 nm from the peak wavelength. Most preferably, the LEDs emit EM with a peak wavelength of about 660 nm and a bandwidth of no greater than +/−30 nm from the peak wavelength, which is particularly effective in energizing skin cells and stimulating skin tissue in the deeper dermis layer.

The LEDs as described hereinabove are preferably incorporated into a hand held device that includes a head and a handle. More preferably, such a hand held device contains a proximity sensor for sensing proximity of the device to the skin surface and signaling the LEDs to active or deactivate in accordance with the proximity of the device of the skin surface. More detail on such hand held LED device can be found in U.S. Patent Application Publication Nos. 2007-185553 and 2006-0093561 and International Patent Application No. WO2006/012752, the contents of which are incorporated herein by reference in their entireties for all purposes.

The topical composition as used in the present invention may comprise one or more skin rejuvenating agents selected from the group consisting of creatine, adenosine phosphate, acetyl carnitine, acetyl hexapeptide, and combinations thereof. If creatine is present, it is preferably present in an amount ranging from about 0.01% to about 40% by total weight of the topical composition, more preferably from about 0.05% to about 20% by weight, and most preferably from about 0.1% to about 5% by weight. If adenosine phosphate is present, it is preferably present in an amount ranging from about 0.01% to about 40% by total weight of the topical composition, more preferably from about 0.05% to about 20% by weight, and most preferably from about 0.1% to about 5% by weight. If acetyl carnitine is present, it is preferably present in an amount ranging from about 0.001% to about 20% by total weight of the topical composition, more preferably from about 0.01% to about 10% by weight, and most preferably from about 0.05% to about 5% by weight. If acetyl hexapeptide is present, it is preferably present in an amount ranging from about 0.001% to about 20% by total weight of the topical composition, more preferably from about 0.01% to about 10% by weight, and most preferably from about 0.05% to about 5% by weight.

The topical composition of the present invention may also comprise one or more skin whitening agents, DNA repair-boosting agents, anti-inflammatory agents, collagen synthesis-enhancing agents, and the like. For example, the topical composition may comprise one or more skin whitening agents selected from the group consisting of ascorbyl glycoside, Trametes versicolor extract, and Glycyrrhiza glabra (licorice) root extract. Suitable DNA repair-boosting agents that can be used in the present invention can be selected from the group consisting of bifida ferment extract and sodium ribonucleic acid. Suitable anti-inflammatory agents that can be used in the present invention can be selected from the group consisting of bisabolol, Glycyrrhiza glabra (licorice) root extract, caffeine, Cola acuminata extract, and ascorbyl glucoside. Suitable collagen synthesis-enhancing agents for the practice of the present invention can be selected from the group consisting of acetyl hexapeptide-8, palmitoyl oligopeptide, retinyl palmitate, and Siegesbeckia orientalis extract. The above-described skin care actives may be present in the topical composition of the present invention at amounts ranging from about 0.001% to about 20% by total weight of the topical composition, more preferably from about 0.01% to about 10% by weight, and most preferably from about 0.05% to about 5% by weight.

In order to fully effectuate the skin benefits derived from the combined use of the skin-rejuvenating topical composition and the LEDs, the skin-rejuvenating topical composition is formulated to achieve a particularly high optical transparency with respect to the EM emitted by the LEDs, so as to allow maximum amount of the EM to be transmitted therethrough onto the skin surface. Specifically, the topical composition of the present invention is capable of transmitting at least 80%, preferably at least 85%, and more preferably at least 90%, of the EM emitted by the LEDs to the skin. In other words, the topical composition absorbs, reflects, scatters, or blocks little or no EM emitted by the LEDs, thereby minimizing any potential interference thereof with the LEDs.

The topical composition of the present invention may further contain one or more additional skin care ingredients for further improving the efficacy of the skin rejuvenating agents and the EM emitted by LED in reducing the appearance of fine lines and wrinkles and improving the skin tone. Such additional ingredients may include, but are not limited to: oils, surfactants, humectants, botanical extracts, vitamins, antioxidants, sunscreen agents, preservatives, and the like. The topical composition may be in the form of an emulsion, gel, suspension, aqueous solution, or in the anhydrous form. If present in the form of an emulsion, the composition may be in the form of a water-in-oil or oil-in-water emulsion. Preferably, the topical composition of the present invention is in the form of an oil-in-water emulsion, with a suggested amount of water ranging from about 10% to about 90%, more preferably from about 20% to about 70%, and most preferably from about 40% to about 60%. If the topical composition is present in the anhydrous form, it may also contain one or more oils, and if so, suggested ranges are from about 1 to 95% by weight of the total composition.

Suitable oils include materials also known as skin conditioning agents such as nonvolatile silicones, esters, paraffinic hydrocarbons, vegetable oils, and synthetic oils. The term “nonvolatile” as used herein means that the compound has a vapor pressure of less than about 2 mm of mercury at 20° C. Preferably, the skin conditioning agent is characterized by a viscosity from about 5 to 10 centistokes at 25° C. up to about 1,000,000 centipoise at 25° C. Particularly preferred are the nonvolatile silicones, including but not limited to: amine functional silicones such as amodimethicone, phenyl substituted silicones such as bisphenylhexamethicone, trimethylsiloxyphenyl dimethicone, phenyl trimethicone, polyphenylmethylsiloxane, dimethicone, phenyl dimethicone, diphenyl dimethicone, and dimethicone substituted with C₂₋₃₀ alkyl groups such as cetyl dimethicone. Suitable esters include mono-, di-, or triesters. Monoesters are in the general form RCO—R′ wherein R and R′ are each independently a C₁₋₄₅ straight or branched chain, saturated or unsaturated alkyl. Diesters may be formed by the reaction of a C₁₋₄₅ aliphatic or aromatic mono- or dihydric alcohol with a C₁₋₄₅ aliphatic or aromatic mono- or dicarboxylic acid, as appropriate, where the aliphatic group may be straight or branched chain, or saturated or unsaturated. Suitable triesters include the reaction products of a C₁₋₄₅ aliphatic or aromatic alcohol having at least three hydroxyl groups with a C₁₋₄₅ carboxylic acid, or C₁₋₄₅ aliphatic or aromatic alcohols with a C₁₋₄₅ tricarboxylic acid, with the aliphatic chains being linear or branched, saturated or unsaturated. Examples include esters of caprylic and capric acids and glycerin such as caprylic/capric triglycerides; esters of glycerin or polyglycerin and stearic acid such as glyceryl stearate, diglyceryl diisostearate; esters of malic acid and isostearyl alcohol such as diisostearyl malate; coco caprylate caprate and the like.

Humectants which may be used in the topical composition of the invention and include glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Preferably, the humectants used in the composition of the invention are C₁₋₆, preferably C₂₋₄ alkylene glycols, most particularly butylene glycol. If present, such humectants may range from about 0.001% to about 25%, preferably from about 0.005% to about 20%, more preferably from about 0.1% to about 15%, by total weight of the topical composition.

Suitable botanical extracts that may be used in the topical composition of the invention include extracts from plants (herbs, roots, flowers, fruits, seeds) such as flowers, fruits, vegetables, and so on, including yeast ferment extract, Padica pavonica extract, Thermus thermophilis ferment extract, Camelina sativa seed oil, Boswellia serrata extract, olive extract, Arabidopsis thaliana extract, Acacia dealbata extract, Acer saccharinum (sugar maple), acidophilus, acorns, aesculus, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza Glabra, Salix Nigra, Macrocycstis Pyrifera, Pyrus Malus, Saxifraga Sarmentosa, Vilis Vinifera, Morus Nigra, Scutellaria Baicalensis, Anthemis Nobilis, Salvia Sclarea, Rosmarinus Officianalis, Citrus Medica Limonum, Panax Ginseng, and mixtures thereof. If present, the amount of botanical extracts preferably ranges from about 0.0001% to about 10%, preferably about 0.0005% to about 8%, more preferably about 0.001% to about 5%, by total weight of the topical composition.

Sunscreen agents that can be used in the topical composition of the present invention include, but are not limited to: benzophenones and derivatives thereof (e.g., benzophenone-3, dioxybenzone, sulisobenzone, octabenzone, hydroxy- and/or methoxy-substituted benzophenones, and benzophenonesulfonic acids and salts thereof); salicylic acid derivatives (e.g., ethylene glycol salicylate, triethanolamine salicylate, octyl salicylate, homomethyl salicylate, and phenyl salicylate); urocanic acid and derivatives thereof (e.g., ethyl urocanate); p-aminobenzoic acid (PABA) and derivatives thereof (e.g., ethyl/isobutyl/glyceryl esters thereof and 2-ethylhexyl p-dimethylaminobenzoate, which is also referred to as octyldimethyl PABA); anthranilates and derivatives thereof (e.g., o-amino-benzoates and various esters of amino-benzoic acid); benzalmalonate derivatives; benzimidazole derivatives; imidazolines; bis-benzazolyl derivatives; dibenzoylmethanes and derivatives thereof (e.g., 4-tert-butyl-4′-methoxydibenzoylmethane, which is commonly referred to as “avobenzone,” and 4-isopropyl-dibenzoylmethane); benzoxazole, benzodiazole, benzotriazoles, and derivatives thereof (e.g., 2-(2-hydroxy-5-methylphenyl) benzotriazole and methylene bis-benzotriazolyl tetramethylbutylphenol, which is commonly referred to as “Tinosorb M”); diphenylacrylates and derivatives thereof (e.g., 2-ethylhexyl-2-cyano-3,3-diphenylacrylate, which is commonly referred to as “octocrylene,” and ethyl-2-cyano-3,3-diphenylacrylate, which is commonly referred to as “etocrylene”); diesters or polyesters containing diphenylmethylene or 9H-fluorene substitutional groups; 2-phenyl-benzimidazole-5-sulphonic acid (PBSA); 4,4-diarylbutadienes; cinnamates and derivatives thereof (e.g., 2-ethylhexyl-p-methoxycinnamate, octyl-p-methoxycinnamate, umbelliferone, methylumbelliferone, methylaceto-umbelliferone, esculetin, methylesculetin, and daphnetin); camphors and derivatives thereof (e.g., 3-benzylidenecamphor, 4-methylbenzylidenecamphor, polyacrylamidomethyl benzylidenecamphor, benzylidene camphor sulfonic acid, and terephthalylidene dicamphor sulfonic acid); triazines and derivatives thereof (e.g., 2,4-bis-{[4-(2-ethyl-hexyloxy)-2-hydroxy]-phenyl}-6-(4-methoxyphenyl)-1,3,5-triazine, which is commonly referred to as “Tinosorb S”); naphthalates and derivatives thereof (e.g., diethylhexyl-2,6-naphthalate); naphtholsulfonates and derivatives thereof (e.g., sodium salts of 2-naphthol-3,6-disulfonic and 2-naphthol-6,8-disulfonic acids); dibenzalacetone and benzalacetonephenone; diphenylbutadienes and derivatives thereof; di-hydroxynaphthoic acid and salts thereof; o- and p-hydroxybiphenyldisulfonates; coumarin derivatives (e.g., 7-hydroxy, 7-methyl, and 3-phenyl derivatives thereof); azoles/diazoles/triazoles and derivatives thereof (e.g., 2-acetyl-3-bromoindazole, phenyl benzoxazole, methyl naphthoxazole, and various aryl benzotriazoles); quinine and derivatives thereof (e.g., bisulfate, sulfate, chloride, oleate, and tannate salts thereof); quinoline and derivatives thereof (e.g., 2-phenylquinoline and 8-hydroxyquinoline salts); tannic acid and derivatives thereof (e.g., hexaethylether derivatives thereof); hydroquinone and derivatives thereof; uric acid and derivatives thereof; vilouric acid and derivatives thereof, and mixtures or combinations thereof. Salts and otherwise neutralized forms of certain acidic sunscreens from the list hereinabove are also useful herein. Particularly preferred sunscreen agents for the present invention are: 4,4′-t-butylmethoxy-dibenzoylmethane, 2-ethylhexyl-2-cyano-3,3-diphenylacrylate, 2-ethylhexylsalicylate, 3,3,5-trimethylcyclohexylsalicylate, 2-ethylhexyl p-methoxycinnamate, 2-hydroxy-4-methoxybenzophenone, 2,2-dihydroxy-4-methoxybenzophenone, 2,4-bis-{4-(2-ethyl-hexyloxy)-2-hydroxy]-phenyl}-6-(4-methoxyphenyl)-1,3,5-triazine, methylene bis-benzotriazolyl tetramethylbutylphenol, terephthalylidene dicamphor sulfonic acid, diethylhexyl 2,6-naphthalate, digalloyltrioleate, ethyl 4-[bis(hydroxypropyl)]aminobenzoate, glycerol p-aminobenzoate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl-sulfoniobenzoxazoic acid, and mixtures or combinations thereof. The above-described sunscreen agents may be used alone or in combination of two or more. In addition, other known animal or vegetable extracts having ultraviolet light-absorbing ability may properly be used alone or in combination. If present, the amount of sunscreen agents preferably ranges from about 0.001% to about 50%, preferably about 0.01% to about 10%, more preferably about 1% to about 5%, by total weight of the topical composition.

The topical composition of the present invention may further contain vitamins and/or antioxidants. Suitable vitamins may include ascorbic acid and derivatives thereof, such as ascrobyl palmitate; the B vitamins such as thiamine, riboflavin, pyridoxin, and the like; Vitamin A and the ester-based derivatives thereof, such as palmitate, acetate, and the like, as well as Vitamin A in the form of beta carotene; Vitamin E and derivatives thereof, such as Vitamin E acetate, nicotinate, or other esters thereof; Vitamins D and K; coenzymes such as thiamine pyrophoshate, flavin adenin dinucleotide, folic acid, pyridoxal phosphate, tetrahydrofolic acid, and the like. Suitable antioxidants may include potassium sulfite, sodium bisulfite, sodium erythrobate, sodium metabisulfite, sodium sulfite, propyl gallate, cysteine hydrochloride, butylated hydroxytoluene, butylated hydroxyanisole, and so on. If present, the amount of vitamins and/or antioxidants may each range from about 0.001% to about 10%, preferably from about 0.01% to about 8%, more preferably from about 0.05% to about 5%, by total weight of the topical composition.

The topical composition may also contain one or more surfactants, particularly if present in the emulsion form. Preferably such surfactants are nonionic and may be in the form of silicones or organic nonionic surfactants. Suggested ranges are from about 0.1 to 40%, preferably from about 0.5 to 35%, more preferably from about 1 to 30% by weight of the total composition. Suitable silicone surfactants include polyorganosiloxane polymers that have amphiphilic properties, for example contain hydrophilic radicals and lipophilic radicals. These silicone surfactants may be liquids or solids at room temperature. Exemplary silicone surfactants that can be used in the present invention include, but are not limited to: dimethicone copolyols, alkyl dimethicone copolyols, and emulsifying silicone elastomers. Emulsifying silicone elastomers are elastomers that have one or more hydrophilic groups such as hydroxyl, oxyethylene, and the like bonded thereto so as to confer hydrophilic properties to the elastomer. Suitable organic nonionic surfactants may include alkoxylated alcohols or ethers formed by the reaction of an alcohol with a polyalkyleneoxide containing repeating units of alkylene oxide. Preferably, the alcohol is a fatty alcohol having 6 to 30 carbon atoms. Examples of organic nonionic surfactants that can be used in the present invention include, but are not limited to: steareth 2-100, beheneth 5-30, ceteareth 2-100, ceteth 1-45, and the like, which are formed by polyethyleneoxide with the corresponding stearyl/behenyl/cetyl alcohol (wherein the number as used herein designates the number of repeating units of ethylene oxide in the polyethyleneoxide). Other alkoxylated alcohols include esters formed by reaction of polymeric alkylene glycols with glyceryl fatty acid, such as PEG glyceryl oleates, PEG glyceryl stearate; or PEG polyhydroxyalkanotes such as PEG dipolyhydroxystearate wherein the number of repeating ethylene glycol units ranges from 3 to 1000. Also suitable as nonionic surfactants are formed by the reaction of a carboxylic acid with an alkylene oxide or with a polymeric ether. Monomeric, homopolymeric, or block copolymeric ethers, alkoxylated sorbitan, alkoxylated sorbitan derivatives can also be used as nonionic surfactants in the present invention.

The topical composition of the invention may also contain other ingredients such as structuring agents in the form of polymeric structuring agents such as acrylic polymers, polyamides or polyurethanes. The structuring agents may be water or oil soluble or dispersible. Such structuring agents will provide structure, or increase the viscosity of the composition. If present, suggested ranges are from about 0.1 to 50%, preferably from about 0.5 to 40%, more preferably from about 1 to 35% by weight of the total composition. Suitable structuring agents include natural, synthetic waxes, or mineral waxes such as petrolatum, candelilla, ozokerite, synthetic wax, polyethylene, and so on. Suitable polymeric structuring agents include acrylic polymers such as carbopol or pemulen (polymers of acrylic acid, methacrylic acid, or their simple esters crosslinked by polyfunctional agents such as allyl ethers of sucrose or pentaerythritol), ester or amide terminated polyamides such as those sold by Arizona Chemical under the Uniclear or Sylvaclear trademarks, or aqueous dispersions or solutions of polyurethanes.

In the case where the topical composition of the invention is colored, from about 0.1 to 80%, more preferably from about 0.5 to 75%, more preferably from about 1 to 70% by weight of the total composition of particulates may be present. The term “particulates” refers to pigments in the form of inorganic or organic pigments such as iron oxides (black, blue, red, yellow), or the D&C and FD&C Lakes. Particulates may also include ingredients commonly referred to as “powders” that is particulate materials that are present for muting color (such as titanium dioxide) or providing bulk to the composition. Further examples include nylon, polymethylmethacrylate, silica, silica silylate, and the like.

The ingredients as described hereinabove are preferably provided in a topical composition that may be formulated into a cream, gel, lotion, oil, ointment, powder, stick, cake, or other forms that can be topically applied. The resulting topical composition may be in the form of a liquid, solid, semi-solid, dispersion, suspension, solution or emulsion, and it can be either aqueous-based or anhydrous. The topical composition of the invention may also be in the form of color cosmetic compositions, such as foundation makeup, blush, and the like. Typical skin creams or lotions comprise from about 5-98% water, 1-85% oil, and from about 0.1 to 20% of one or more surfactants. Typical color cosmetic compositions such as foundations, blush, and the like may be in the anhydrous or aqueous form. If aqueous based, such compositions will preferably contain from about 5-98% water, 1-85% oil, and optionally from about 0.1 to 20% of one or more surfactants in addition to from about 0.1 to 65% of particulates that are pigments or a combination of pigments and powders. If anhydrous, the compositions may contain from about 0.1 to 95% oil, from about 0.1 to 99% particulates, and optionally from about 0.1 to 50% of one or more structuring agents. Typical toner compositions comprise from about 0.1 to 99% of water or other polar nonaqueous solvent such as ethanol, propylene glycol, butylene glycol. Toners are typically applied for cleansing purposes using a cotton pad or other applicator to swipe across the skin to remove debris or dirt. Typical spritzer compositions include those that may be sprayed on the skin. Preferably such compositions will contain from about 0.1 to 99% of water or other polar nonaqueous solvent. Such compositions are generally applied as leave on compositions. Typical gels are aqueous based and may contain from about 0.1 to 95% water, from about 0.1 to 50% structuring agents.

The invention will be further described in connection with the following examples which are set forth for the purposes of illustration only.

### EXAMPLES

### Example 1

### Skin-Rejuvenating Serums

| | Wt % | |
|---|---|---|
| Components | Formula 1 | Formula 2 |
| De-ionized water | QS | QS |
| Bifida ferment lysate | 9.40 | 9.40 |
| PEG-75 | 4.00 | 4.00 |
| Methyl gluceth-20 | 4.00 | 4.00 |
| Butylene glycol/water/Cola acuminata (kola) | — | 3.00 |
| extract | | |
| Water/bifida ferment lysate/hydrogenated lecithin | 3.00 | 3.00 |
| Bis-PEG-18 methyl ether dimethyl silane | 2.00 | 2.00 |
| Butylene glycol | 1.33 | 1.33 |
| Glycereth-26 | 1.00 | 1.00 |
| Phenoxyethanol | 0.72 | 0.72 |
| Triethanolamine | 1.61 | 0.61 |
| Squalane | 0.50 | 0.50 |
| Oleth-3 phosphate | 0.45 | 0.45 |
| Oleth-3 | 0.35 | 0.35 |
| Methylparaben | 0.03 | 0.33 |
| Carbomer | 0.26 | 0.26 |
| Oleth-5 | 0.24 | 0.24 |
| Pantethine | 0.14 | 0.14 |
| Retinyl palmitate/corn oil/BHT/BHA | 0.10 | 0.10 |
| Trisodium EDTA | 0.10 | 0.10 |
| BHT | 0.10 | 0.10 |
| Bisabolol | 0.10 | 0.10 |
| Choleth-24/ceteth-24 | 0.10 | 0.10 |
| Ethylhexyl methoxycinnamate | 0.10 | 0.10 |
| Benzyl alcohol | 0.10 | 0.10 |
| Xanthan gum | 0.08 | 0.08 |
| Caffeine | 0.20 | 0.05 |
| Chamomile | — | 0.03 |
| Sodium ribonucleic acid | 0.10 | 0.01 |
| Sodium hyaluronate | 0.01 | 0.01 |
| Ascorbyl glucoside | 2.00 | — |
| Siegesbeckia orientalis extract/glycerin | 1.00 | 0.50 |
| Glyceryl polymethacrylate/PEG-8/palmitoyl | 0.20 | 0.20 |
| oligopeptide | | |
| Potassium sorbate | 0.10 | — |
| Water/acetyl hexapeptide-8 | 0.10 | 0.10 |
| Water/butylene glycol/lecithin/caprylyl | 0.10 | — |
| glycol/hexylene glycol/hydroxyethylcellulose/ | | |
| acetyl hexapeptide-3/hydrolyzed fish | | |
| collagen/lauryldimonium hydroxypropyl | | |
| hydrolyzed soy protein | | |
| Chamomilla recutita extract | 0.10 | 0.05 |
| Creatine | 0.10 | 0.08 |
| Yeast extract | 0.10 | 0.10 |
| Acetyl carnitine HCl | 0.10 | 0.001 |
| Adenosine phosphate | 0.10 | 0.05 |
| Glycyrrhiza glabra (licorice) root extract | 0.05 | — |
| Malt extract | 0.01 | — |
| Black strap molasses | 0.01 | — |
| Pigments | 0.0014 | 0.0014 |

The light transmission rates of Formulas 1 and 2 were measured by a Hewlett-Packard UV-Vis spectroscope (Model HP 8452A) upon application to a 0.1 mm path length quartz cell. Each sample material was placed in the cell, which was then placed in the UV-Vis spectroscope instrument to measure the amount of light transmitted through the sample from the LED source to the detector. Results are recorded in terms of intensity vs wavelength. Formula 1 demonstrated a light transmission rate of about 92%, and Formula 2 demonstrated a light transmission rate of about 88%.

### Example 2

### Improved Wrinkle Reduction Results Achieved by the Combined Used of Serum Formulation 1 and LED

Formulation 1 as described hereinabove in Example 1 was used in combination with a hand held 660 nm red light LED skin care device commercially available from Pharos Life Corporation in Ontario, Canada as Tända Regenerate™ device. Specifically, Formulation 1 was applied to the entire face first. Next, the Tända Regenerate™ device was used to treat the right and left canthus for three (3) minutes each, and then three forehead regions for one (1) minute each. The Tända Regenerate™ device was held directly onto the skin surface and held still for the respective treatment time. Every thirty (30) seconds the Tända Regenerate™ device beeped and then automatically shut off once each three (3) minute treatment time period expired. Such combined treatment was repeated by each subject two times daily during the entire study period, which was eight (8) weeks, and no other moisturizers or treatment products were used for the duration of the study. Baseline measurements were first taken as control before the treatment started, and subsequent measurements were then taken at four (4) weeks and the end of the eight (8) weeks. For comparative purposes, the skin rejuvenating results achieved by using Formulation alone and the Tända Regenerate™ device alone were also measured.

For each measurement, the subjects first acclimated in an environmental room at 40% relative humidity and ambient temperature (70° F.) for 20 minutes upon arrival at the testing center. Following the acclimation period, the forehead lines of each subject were measured via photography, and the canthus lines and wrinkles were measured via replicas, as described in greater detail hereinafter.

Photographic measurement of the forehead lines was conducted using Canfield's VISIA-CR™ photo-imaging equipment, which produced high quality, reproducible facial images that could then be analyzed through clinical evaluations of various skin features. The facial imaging booth minimized variability in images captured at different intervals, allowing for comparative assessments of changes in facial features over the course of time.

The Canfield Facial Imaging Booth with VISIA-CR™ consisted of a fixed head support and image preview tools to ensure proper re-positioning of each subject from baseline to endpoint. The system had multiple built in lighting modes and could acquire up to seven images in one sitting from user-definable shooting templates. Subjects could be photographed using standard light, UV, cross-polarization, parallel-polarization, or any combination of these to enhance visualization of designated skin features. The camera could be rotated 180° around the head, and there were lock stops along the rotation to fix the camera position.

Photographs were taken by reproducibly positioning the head of the subject, using stationary chin and forehead supports and maintaining consistent camera and lighting settings at each study visit. Images captured with the VISIA-CR™ were saved directly to an electronic record in Canfield's Mirror software.

The captured images were then evaluated via an image analysis program, Image Pro 6.0, comparing before and after product use. The images were first cropped to the area of interest (AOI) that was to be analyzed. The dimensions of the AOI were stored so that the same AOI was used for all time points throughout the study to maintain reproducibility in this analysis phase.

The images were then automatically processed by the Image Pro 6.0 program using the RGB color analysis to perform image segmentation to identify the lines and wrinkles Image segmentation was the method by which certain parts of a digital image are isolated based on particular colors in that image. Forehead lines appeared as darker colors whereas the surrounding skin appeared lighter. Once the feature was isolated, the image was then converted to a composite binary (2 color) image with one color representing the feature and the other color representing everything else in the image.

Based on these binary images, the total number of pixels that represented a feature in a given area was then measured. Subsequent time points were then analyzed in this same manner. If the product has indeed reduced the appearance of forehead lines, the total number of pixels that represented those lines will decrease in subsequent images. Finally, an overall percentage change between the baseline time point image and the final end point image for all the test subjects in the study was calculated.

Replica evaluation of the canthus lines and wrinkles were carried out by first placing adhesive rings on each canthus, then placing a dental silicone replicating material, SILFLO (available from Flexico in England), thereover to make a silicone replica. Approximately five (5) grams of SILFLO were poured into an aluminum dish, and several drops of drops of catalyst were added. The mixture of SILFLO and catalyst was vigorously stirred and spread inside the rings. As soon as the silicone dried (about 2-3 minutes), the replicas were removed and labeled with the panelist's name and visit. One set of replicas was collected at each visit. At the end of the study, the replicas were analyzed via digital image analysis for lines and wrinkles.

Each replica was placed at the same point beneath a Panasonic CCD black and white camera and illuminated with a Nikon fiber optic light source at a fixed low angle. The camera was interfaced to the Zeiss KS400 imaging system which analyzes each replica. Fine lines and wrinkles are assessed by examining changes in the Integrated Optical Density (IOD) before and after product use, which were automatically outputted by the Panasonic CCD camera. A decrease in IOD represented a decrease in fine lines and wrinkles and vise-versa.

It was demonstrated that after 4 weeks of combined treatment by the Tända Regenerate™ device and Formulation 1, an average wrinkle reduction of about 42% was achieved, which was statistically significantly higher than the 34% reduction achieved by treatment using the Tända Regenerate™ device alone and the 37% reduction achieved by treatment using Formulation 1 alone. After 8 weeks of combined treatment, an average wrinkle reduction of about 54% was achieved, which was statistically significantly higher than the 40% reduction achieved by treatment using the Tända Regenerate™ device alone and the 45% reduction achieved by treatment using Formulation 1 alone.

### Example 3

### Improved Wrinkle Reduction Results Achieved by the Combined Used of Serum Formulation 2 and LED

Formulation 2 was also used in combination with the Tända Regenerate™ device to treat the fine lines and wrinkles on the forehead and left and right canthus, following the same testing methods described hereinabove in Example 2.

It was demonstrated that after 4 weeks of combined treatment by the Tända Regenerate™ device and Formulation 2, an average wrinkle reduction of about 43% was achieved, which was statistically significantly higher than the 34% reduction achieved by treatment using Formulation 2 alone. After 8 weeks of combined treatment, an average wrinkle reduction of about 52% was achieved, which was statistically significantly higher than the 41% reduction achieved by treatment using Formulation 2 alone.

### Example 3

### Improved Skin Tone Evenness Achieved by the Combined Used of Serum Formulation 1 and LED

The effectiveness of the Tända Regenerate™ device in combination with Formulation 1 in improving skin tone evenness was also measured. Specifically, Formulation 1 was applied to the entire face first. Next, the Tända Regenerate™ device was used to treat the right and left canthus for three (3) minutes each, and then three forehead regions for one (1) minute each. The Tända Regenerate™ device was held directly onto the skin surface and held still for the respective treatment time. Every thirty (30) seconds the Tända Regenerate™ device beeped and then automatically shut off once each three (3) minute treatment time period expired. Such combined treatment was repeated by each subject two times daily during the entire study period, which was eight (8) weeks, and no other moisturizers or treatment products were used for the duration of the study. Baseline measurements were first taken as control before the treatment started, and subsequent measurements were then taken at four (4) weeks and the end of the eight (8) weeks. For comparative purposes, the skin tone evenness achieved by using Formulation alone and the Tända Regenerate™ device alone were also measured.

For each measurement, the subjects first acclimated in an environmental room at 40% relative humidity and ambient temperature (70° F.) for 20 minutes upon arrival at the testing center. Following the acclimation period, a particular area of approximately 1 cm² on the cheek of each subject was marked for skin tone measurement. The images of that particular area of the face were then captured by a Hi-Scope™ fiber optic microscope (Model KH-1000) manufactured by Hirox (Toyko, Japan) at a 20× magnification. Three (3) images were recorded for each area. The stored images were digitized and analyzed using Optimas 6.51 image analysis software (Media Cybernetics, Carlsbad, Calif.). Specifically, the Grey value of each of the three color channels (i.e., red, green, and blue) of each pixel on each stored images was determined, and the average Grey value and standard deviation of each of the three color channels were then calculated. The standard deviation is an indicator of the amount of color variation in each image. If a product was effective in evening the skin tone, then there would be a significant degree in the standard variation of the Grey value so calculated.

It was demonstrated that after 4 weeks of combined treatment by the Tända Regenerate™ device and Formulation 1, an average increase of about 24% in skin tone evenness was achieved, which was statistically significantly higher than the 18% increase achieved by treatment using the Tända Regenerate™ device alone and the 19% increase achieved by treatment using Formulation 1 alone. After 8 weeks of combined treatment, an average increase of about 30% in skin tone evenness was achieved, which was statistically significantly higher than the 21% increase achieved by treatment using the Tända Regenerate™ device alone and the 24% increase achieved by treatment using Formulation 1 alone.

### Example 4

### Improved Skin Tone Evenness Achieved by the Combined Used of Serum Formulation 2 and LED

The effectiveness of the Tända Regenerate™ device in combination with Formulation 2 in improving skin tone evenness was also measured using the same method as described hereinabove in Example 3.

It was demonstrated that after 8 weeks of combined treatment by the Tända Regenerate™ device and Formulation 1, an average increase of about 23% in skin tone evenness was achieved, which was statistically significantly higher than the 17% increase achieved by treatment using Formulation 2 alone.

What is claimed is:

1. A system for rejuvenating the skin, reducing the appearance of fine lines and wrinkles, and improving the skin tone comprising:
(a) a topical composition comprising one or more skin rejuvenating agents selected from the group consisting of creatine, adenosine phosphate, acetyl carnitine, acetyl hexapeptide, and combinations thereof in a pharmaceutically or cosmetically acceptable carrier; and
(b) a phototherapy device comprising one or more light emitting diodes (LEDs) for emitting electromagnetic radiation effective for energizing the skin cells and enhancing skin absorption of the skin rejuvenating agents,
wherein the topical composition is capable of transmitting at least 80% of the electromagnetic radiation emitted by the phototherapy device to the skin.

2. The system of claim 1, wherein the topical composition is an oil-in-water emulsion comprising creatine, adenosine phosphate, acetyl carnitine, and acetyl hexapeptide.

3. The system of claim 1, wherein the topical composition is capable of transmitting at least 90% of the electromagnetic radiation emitted by the phototherapy device to the skin.

4. The system of claim 1, wherein the electromagnetic radiation emitted by the phototherapy device is characterized by a peak wavelength ranging from about 570 nm to about 850 nm.

5. The system of claim 4, wherein the peak wavelength is selected from the group consisting of about 580 nm, 630 nm, 633 nm, 660 nm, and 830 nm.

6. The system of claim 5, wherein the electromagnetic radiation emitted by the phototherapy device is characterized by a bandwidth of no greater than +/−40 nm from the peak wavelength.

7. A unit package comprising:
(a) a container comprising a topical composition for application to the skin, wherein said topical composition comprises one or more skin rejuvenating agents selected from the group consisting of creatine, adenosine phosphate, acetyl carnitine, acetyl hexapeptide, and combinations thereof in a pharmaceutically or cosmetically acceptable carrier; and
(b) a phototherapy device comprising one or more light emitting diodes (LEDs) for emitting electromagnetic radiation effective for energizing the skin cells and enhancing skin absorption of the skin rejuvenating agents,
wherein the topical composition is capable of transmitting at least 80% of the electromagnetic radiation emitted by the phototherapy device to the skin.

8. The unit package of claim 7, wherein the topical composition is an oil-in-water emulsion comprising creatine, adenosine phosphate, acetyl carnitine, and acetyl hexapeptide.

9. The unit package of claim 7, wherein the topical composition is capable of transmitting at least 90% of the electromagnetic radiation emitted by the phototherapy device to the skin.

10. The unit package of claim 7, wherein the electromagnetic radiation emitted by the phototherapy device is characterized by a peak wavelength ranging from about 570 nm to about 850 nm.

11. The unit package of claim 10, wherein the peak wavelength is selected from the group consisting of about 580 nm, 630 nm, 633 nm, 660 nm, and 830 nm.

12. The system of claim 11, wherein the electromagnetic radiation emitted by the phototherapy device is characterized by a bandwidth of no greater than +/−40 nm from the peak wavelength.

13. A method for rejuvenating the skin, reducing the appearance of fine lines and wrinkles, and improving the skin tone comprising:
(a) applying to the skin a topical composition comprising one or more skin rejuvenating agents selected from the group consisting of creatine, adenosine phosphate, acetyl carnitine, acetyl hexapeptide, and combinations thereof in a pharmaceutically or cosmetically acceptable carrier; and
(b) radiating the skin with electromagnetic radiation emitted from one or more light emitting diodes (LEDs), wherein said electromagnetic radiation is effective for energizing the skin cells and enhancing skin absorption of the skin rejuvenating agents,
wherein the topical composition is capable of transmitting at least 80% of the electromagnetic radiation emitted by the LEDs to the skin.

14. The method of claim 13, wherein steps (a) and (b) are carried out sequentially.

15. The method of claim 13, wherein steps (a) and (b) are carried out simultaneously.

16. The method of claim 13, wherein the topical composition is an oil-in-water emulsion comprising creatine, adenosine phosphate, acetyl carnitine, and acetyl hexapeptide.

17. The method of claim 13, wherein the topical composition is capable of transmitting at least 90% of the electromagnetic radiation emitted by the phototherapy device to the skin.

18. The method of claim 13, wherein the electromagnetic radiation emitted by the phototherapy device is characterized by a peak wavelength ranging from about 570 nm to about 850 nm.

19. The method of claim 18, wherein the peak wavelength is selected from the group consisting of about 580 nm, 630 nm, 633 nm, 660 nm, and 830 nm.

20. The method of claim 19, wherein the electromagnetic radiation emitted by the phototherapy device is characterized by a bandwidth of no greater than +/−40 nm from the peak wavelength.
